(19) United States
(12) Patent Application Publication
(10) Pub. No.: US 2024/0212817 A1
(43) Pub. Date: Jun. 27, 2024

(54) SYSTEM AND METHOD FOR MONITORING PACKAGE OPENING AND PERSONALIZING USER ENGAGEMENT

(71) Applicant: Synchronyx, LLC, Boca Raton, FL (US)

(72) Inventor: Tamar SAPIR, Boca Raton, FL (US)

(73) Assignee: Synchronyx, LLC, Boca Raton, FL (US)

(21) Appl. No.: 18/412,568

(22) Filed: Jan. 14, 2024

Related U.S. Application Data
(63) Continuation-in-part of application No. 17/919,268, filed on Oct. 16, 2022, now abandoned.
(60) Provisional application No. 62/704,203, filed on Apr. 27, 2020.

(51) Int. Cl. G16H 20/10; G09F 3/00; H04L 67/55

(52) U.S. Cl. CPC G16H 20/10; G09F 3/0297; H04L 67/55

(57) ABSTRACT

A system and method for real-time tracking, monitoring, and engagement of users with regard to opening of packaged goods and personalization of user engagement through the use of encoded labels and a cloud-based platform is disclosed. The system and method can be used for event monitoring and in particular for medical therapy management. Medication adherence can be reported in real-time and personalized notices and reminders can be sent to users to remind them to take medication as prescribed and link them to relevant resources.

### FIELD OF THE INVENTION

The invention relates to a system and method for real-time tracking, monitoring, and engagement of users with regard to opening of packaged goods and personalization of user engagement through the use of encoded labels and a cloud-based platform. The system and method can be used for event monitoring and in particular for medical therapy management. Medication adherence can be recorded in real-time and notices and reminders can be sent to users to remind them to take medication as prescribed and link them to relevant resources to stay connected with recommended plan of care.

### BACKGROUND OF THE INVENTION

Advancements in communication technologies have made significant improvements in the ability to track and trace packaged goods across various industries, with the ability to provide real-time reporting of exact locations, positioning, arrival/departure times, and transfer of data and status reports on each such goods shipment. Such advancements can be used in tracking and locating shipped packages, containers, food items, pharmaceuticals, agriculture, vehicles, valuable items, merchandise, apparel, and animals.

A range of such tracking and tracing solutions and tools include use of radiofrequency identification (RFID), barcode solutions, GPS and satellite tracking. These solutions can track and report location of shipped packaged goods in real-time in response to a signal and many require a power source to transmit their signal to a reader. Battery-operated readers or hardware can be bulky, obtrusive, and costly, and may not have the ability to record when a packaged good was opened in real time without the use of power source.

Tracking the exact time, date, and geolocation of such events as the opening and use of a packaged good can provide real-time insights and actionable triggers based on consumer behaviors and utility of the packaged goods. This information can predict barriers and address timely solutions in supply chain, inventory, and service delivery logistics to mitigate risk and optimize consumer experience and outcomes. Examples of industries where event monitoring might be of great value and importance may include but are not limited to a multitude of shipping and delivery services, food and beverage, medical supplies and health care.

Particularly, event monitoring may be advantageous in tracking the use of pharmaceuticals by patients according to the prescribed dosage, time, frequency, and direction, also known as medication adherence. Poor medication adherence is a global problem that particularly affects the treatment of chronic diseases and comorbid conditions, which involves polypharmacy and requires long-term administration of medications. In the US alone, 6 of 10 adults live with at least one chronic disease, like heart disease and stroke, cancer, or diabetes. These and other chronic diseases are the leading causes of death and disability in the country and are also a leading driver of the nation's $3.5 trillion in annual health care costs. Patient non-adherence with the taking of prescribed medications is associated with poor therapeutic outcomes, worsening of disease, and onset of comorbid conditions, leading to unnecessary hospitalizations, avoidable doctor and emergency department visits, increased health care service utilization, and overall health care costs. According to the World Health Organization (WHO) over 50% of people suffering from chronic conditions in developed countries are non-adherent to their medications, with even higher rates seen in developing countries. Furthermore, drug-related morbidity and mortality from non-optimized medication therapy have an estimated annual cost of $528.4 billion, equivalent to 16% of total US health care expenditures in 2016. Yet, about 75% of the population faces trouble in taking medications as instructed, more than a fifth of new prescriptions go unfilled, and 33-69% of medication-related hospital admissions occur due to non-adherence. Furthermore, adherence is known to be the lowest among patients with chronic diseases.

Medication therapy management (MTM) programs thus have the potential to improve medication adherence, optimize healthcare outcomes, and reduce healthcare spending. MTM programs also attempt to address complicated dosing regimens derived from the use of multiple medications (polypharmacy) and the need to manage potential drug-drug interactions that are associated with complex dosing schemes. Simplification of dosing regimens coupled with effective counseling and communication is an important factor in ensuring optimal medication consumption and adherence.

Methods that have been utilized for measuring medication adherence can be broadly divided into two categories, direct and indirect. Direct methods of measurement of adherence include direct observation of the patient while taking the medication, laboratory detection of the drug in the biologic fluid of the patient, laboratory detection of the presence of nontoxic markers added to the medication in the biologic fluid of the patient, and laboratory detection of the presence of biomarkers. Direct measures may be accurate, but can be invasive, are usually expensive, and may be limited to only select drugs.

In the clinical setting, most measurements of nonadherence include indirect or subjective methods such as patient self-reported questionnaires and clinician-observed medical record documentation. Indirect measures of nonadherence may sometimes be accurate, but they rely on the reliability of the user which poses risks for selection bias, and excess cost. Furthermore, the data adherence collected from these methods is often retrospective, and do not provide a real-time look at adherence patterns and behaviors.

Pharmacy groups across different settings have incorporated specific forms of drug packaging to optimize medication therapy management solutions. PASS packaging, for example, organizes prescribed medications, over-the-counter oral solids, and even vitamins, by day and dose time in clear sealed plastic pouches or packets. The pre-sorted individual packets are delivered to the patients as part of a monthly supply of their medications. Each packet is labeled with the patient's name, day of the week, date, time of dose, and drug information. Clearly labeled packaging can be an aid to patients who may be forgetful or confused; one can look at the next packet on the strip to confirm that a medication dose was taken.

Efforts to increase medication adherence are increasingly using modern technologies such as the Internet and mobile phones. Sending reminders to patients to take their medication by Short Message Service (SMS) is a simple method with low intrusiveness and relatively low cost that is increasingly being used. A detailed record of drug intake can be generated through real time assessment of the date and time that the box or bottle was opened, offering the opportunity to send a reminder SMS in the event that the patient does not open the box within a certain time frame. Real-time medication monitoring technologies include the use of medication event monitoring systems (MEMS) that may utilize a pill bottle fitted with an electronic cap, in which the cap has a sensor that detects when the bottle is opened. The information is transferred to a battery-operated MEMS reader which, in turn, sends the information to a computer or syncs with an app so that healthcare providers and patients can track medication adherence over time. This technology requires bottles that are large, bulky, and require a power-source for information transfer. Furthermore, the MEMS cap detects when the pill bottle is opened for one type of medication at a time and does not address polypharmacy issues or is not compatible with certain packaging formats such as blister packs or injection boxes.

Some smart technologies track medication adherence from blister packs through deposition of a printed electronics layer onto the manufacturer's packaging postproduction, which allows the smart blister packs to pair to a smart phone through an Android™ or iOS™ application for monitoring of medication use. Similar blister pack solutions for adherence tracking include battery-operated sensors attached to pill sheets that are able to track when pills are taken through monitoring movement, touch, and sound.

Other medication tracking devices rely on technologies such as RFID, Bluetooth™, or other embedded electronics to scan labels on pill bottles or pods that can be “activated” by interaction with an external central hub system such as a WiFi™-enabled transmitter. A central hub system collects data, sends medication reminders, and sends notifications of missed medication doses.

Other techniques for adherence monitoring include ingestible sensors the size of a grain of a sand that are incorporated to medications through a digital health feedback system featuring an ingestible event marker (IEM) which, once activated by stomach acid, can transmit physiological information to a monitoring patch worn on the patients' torso, which in turn sends the information to a computer.

U.S. Patent Appln. Publn. No. US20180096175 to Schmeling et al. and titled BLOCKCHAIN ENABLED PACKAGING discloses blockchain enabled packaging to track movement and conditions of packages from manufacturers through to delivery. Any or all transactions performed via or in relation to a distributed manufacturing platform may be recorded to a ledger or blockchain, capturing real-time information about the package and its contents.

U.S. Patent Appln. Publn. No. US20160026773 to SMRxT Inc. and titled METHODS AND SYSTEMS OF REAL-TIME MEDICATION ADHERENCE MONITORING discloses systems and methods for monitoring medication usage and adherence to one or more patients. In one embodiment, a container that holds medication is configured with one or more sensors to sense the medication inside the container, a container processing circuit configured to determine a medication amount within the container based on readings from the one or more sensors and a wireless communication circuit configured to transmit the medication amount over a wireless communication network. The sensor may be an orientation sensor which may be an accelerometer, a gyroscope, a magnetometer, or an analog and digital level. Additional sensors may detect physical characteristics regarding the medicine in the container or environmental conditions. The sensor may detect the weight of the medicine in the container by use of load force applied to a surface, or measure changes in electrical signals based on changes in force, torsion or load placed on a strain gauge. The sensor may detect volume within the container. The sensor may detect motion of the medicine in the container and motion of the container.

U.S. Pat. No. 9,443,276 to Barry and titled EVENT-BASED TRACKING, ORDER ADHERENCE, AND REWARDS MANAGEMENT WITH NFC-ENABLED ELECTRONIC DEVICES discloses an NFC-enabled electronic device comprising a processor, a memory accessible to the processor and a NFC reader under control of the processor with processor-executable instructions that perform asset tracking. In one embodiment, the asset is a medical asset.

U.S. Pat. No. 9,901,515 to Roberts et al. and titled SMART CAP FOR MEDICATION CONTAINER discloses a smart cap for a medication container including a circuit board and a weight sensor electrically coupled to the circuit board and configured to generate electrical input signals representative of the total weight of medication units in the medication container, and a memory for storing medication information associated with medication in the medication container. The smart cap may transmit the stored information to a computing device communicatively coupled to the cap.

U.S. patent Ser. No. 10/282,521 to Alarm.com Incorporated and titled MEDICATION MANAGEMENT AND REPORTING TECHNOLOGY discloses technology in which output from at least one sensor is configured to sense physical activity in a building in which medication of a patient is located is monitored and a determination is made to capture one or more images of the medication based on the monitoring. A camera is used to capture an image of the medication and the captured image is analyzed to detect a state of the medication. Information regarding a schedule by which the medication should be taken by the patient is accessed and an expected state of the medication is determined. The detected state is compared with the expected state and a determination is made that the patient has departed from the schedule based on the comparison revealing that the detected state does not match the expected state. A message indicating the departure from the schedule is sent based on the determination. The sensor may be a motion sensor configured to detect motion in the room in which the medication is located.

U.S. patent Ser. No. 10/282,967 to Avery Dennison Corporation and titled TIME-TEMPERATURE TRACKING LABEL discloses a system including a laminated label and a computer application, where the laminated label tracks and records information associated with a product having a time-temperature threshold. If the time-temperature threshold is exceeded, the product is considered unsuited for its intended purpose. The information recorded by the label is wirelessly communicated from the label to the computer application. The laminated label includes a facestock, a microchip, a communication apparatus, an indicator and a power source.

U.S. patent Ser. No. 10/902,946 to WatchRX, Inc. and titled MEDICATION, ADHERENCE DEVICE AND COORDINATED CARE PLATFORM discloses a method of supporting user adherence to a medication regimen that stores received medication information associated with one or medications using a patient wearable device (PWD) and a coordinated care platform. In one embodiment, the PWD is a wrist-worn apparatus that includes a chronometer, a cellular telephone, a GPS receiver, a camera and one or more sensors such as a gyroscope, accelerometer and/or a magnetometer. The PWD may provide visual and audible medication reminders. The coordinated care platform receives, processes and analyzes information from the PWD.

### SUMMARY OF THE INVENTION

The invention relates to a system and method of real-time tracking, monitoring, and engagement of users with regard to opening of packaged goods and personalization of user engagement through the use of encoded labels and a cloud-based platform. Encoded labels paired with internet-enabled smart devices monitor and report in real-time if, when, and where a packaged good is opened and/or used by a user. Data captured by the smart device are in turn stored in a cloud-based central database that processes and transfers the data to diverse platforms using an Application Programming Interface (API). Through smart logic algorithms pre-set to identify diverse event-based thresholds, the platform triggers notifications, alerts, and outreach via diverse digital and human communication channels.

The invention comprises one or more encoded labels that are “activated” at the time that a user opens a packaged good. A packaged good may contain any type of goods including but not limited to medications, health and wellness supply, consumables, food, beverage, etc., and the packaging may be in any form including but not limited to bottles, blister packs, calendar packs, plastic bags, boxes, tubes, syringes, infusion cards, etc. The encoded labels may be passive or active. A passive encoded label is a passive device that operates without a power supply of its own and is reliant on an active device to come into range before it is activated, through electromagnetic induction of a current in an integrated circuit on the passive device. The passive encoded label may also generate power from low-level energy harvesting sources such as solar, RF, and thermal gradients. Other forms of passive encoded labels may be “read only” and do not require activation to read encoded information. An active encoded label can transmit information to a smart device without the need for external power because a power source such as a battery is incorporated into the encoded label. Activation indicates the reading of encoded information that has been encoded in the encoded label by a reader and occurs according to the type of encoded label that is on the packaged good. For example, an NFC label on a packaged good may be “activated” by tapping an NFC-enabled device in the proximity of the NFC encoded label. Upon activation, information contained in the encoded labels that are read by a reader is transmitted by the smart device to a cloud-based platform (the “platform”). Additionally, non-encoded information detected by the smart device, such as date, time, geolocation of package opening/use, as well as other physical properties of the packaged good or its surroundings (weight, rotation, opening, temperature, light, moisture, etc), can also be transmitted to the platform.

The invention further comprises a platform comprising a database and programmable processor, a label encoding interface, a user portal, an admin/provider portal, a communication link and a memory. The platform further comprises a plurality of algorithms that may trigger activation of certain actions based on information received from the smart device.

The invention further comprises a plurality of users, each user having an account stored in the memory of the platform and storing user information. The users are the consumers of the packaged goods.

The invention further comprises a plurality of providers, each provider having an account stored in the memory of the platform and storing provider information. Providers provide the packaged goods to the users directly or indirectly. Providers may provide various services. For example, a provider may be a manufacturer of a good, or may be a provider of any service along the supply chain of a packaged good including a distributor or retailer of the packaged good.

The invention yet further comprises a plurality of user devices, each device comprising an app or web-based software that is enabled to receive information from the encoded label on the packaged good. The type of enablement will depend on the type of encoded label. For example, an NFC-enabled device is provided with application software enabling the device to read information stored on an encoded label. The NFC-enabled device is provided with the antenna of the NFC label that is activated by a carrier field provided by the NFC-enabled device and has a transmission frequency of 13.56 MHz. Information in the NFC-enabled encoded label that is attached to a packaged good provides a unique identifier from which the goods contained in the package to which the encoded label is attached can be identified. The user of the system who receives the packaged good is also identified directly or indirectly by the information read from the NFC-enabled encoded label by the NFC-enabled device. In one embodiment, the unique identifier is associated with a user in the database and programmable processor of the platform which allows identification of the user upon receipt of information from the encoded label.

The invention further comprises predetermined instructions for user outreach associated with users' actions regarding package opening and engagement. In one embodiment, the predetermined instructions are determined by one or more of the providers. The predetermined instructions may be executed based upon information received (or not received) from an activated encoded label. For example, a provider may provide instructions in association with a user relating to a specific encoded label based upon information received from an activated encoded label, presumably by the user. If the time of activation of the encoded label falls within a certain time range, the instruction may be to take no action. However, if the activation of the encoded label falls outside of a certain time range, a message may be automatically sent by the platform through the communications portal to the user, for example reminding the user to open the packaged good and possibly to take some action. In one embodiment, where the packaged good is a medication, the message may instruct the user that it is time to consume the packaged goods. Similarly, failure to receive information within a certain time range may be interpreted as meaning that the encoded label was not activated, meaning the packaged good was not opened. A message may be automatically sent by the platform through the communications link to the user, for example reminding the user to open the packaged good and possibly to take some action. In one embodiment, where the packaged good is a medication, the message may instruct the user that it is time to consume the packaged goods. In one embodiment, the platform can be customized to tier the predetermined instructions based on the frequency and extent of user's package opening and engagement.

### BRIEF DESCRIPTION OF THE DRAWINGS

The invention will be more readily understood from the following detailed description of the various aspects of the invention taken in conjunction with the accompanying drawings in which:

FIGS. 1A-1I depict a variety of packaging and exemplary placement of encoded labels according to various embodiments of the invention.

FIG. 2 depicts a schematic of a platform suitable for use according to one embodiment of the invention comprising a database and programmable processor; a label encoding interface; a user portal; an admin/provider portal; a plurality of algorithms; and a communications portal.

FIGS. 3A-3F depict schematics of the database and programmable processor; the label encoding interface; the user portal; the admin/provider portal; the plurality of algorithms; and the communications portal according to the embodiment of the invention shown in FIG. 2.

FIG. 4 depicts a flow scheme of the execution of exemplary algorithms resident in the database of the platform that can be executed by the database and programmable processor.

FIG. 5 depicts a flow scheme of exemplary actions that may be taken upon confirmation of activation of an encoded label recorded on a user profile according to one embodiment of the invention.

FIGS. 6A-6F depict activation schematics of various encoded labels as used according to one embodiment of the invention.

FIG. 7 depicts a flow scheme of the method according to one embodiment of the invention.

FIGS. 8A-8I depict mock-up screens depicting the platform as viewed through an app resident on a smartphone of a user of the platform according to one embodiment of the invention.

FIG. 9 depicts another embodiment of the invention.

### DETAILED DESCRIPTION OF THE INVENTION

The invention comprises one or more encoded labels that are activated at the time that a user opens a packaged good. A packaged good may comprise any type of goods including but not limited to medications, health and wellness supply, consumables, food, beverage, etc., and may be in any form including but not limited to bottles, blister packs, calendar packs, plastic bags, boxes, tubes, syringes, etc. In one embodiment, an encoded label may be placed directly on the good. Activation occurs according to the type of encoded label that is on the packaged good. Encoded labels may be passive or active.

A passive encoded label operates without a power supply of its own and has information encoded on the label that can be read by a smart device. Some passive encoded labels can be powered if an active device comes into range through electromagnetic induction of a current in an integrated circuit on the passive device. In one embodiment, the passive encoded label generates power from low-level energy harvesting sources such as solar, RF, and thermal gradients. Passive encoded labels that can be activated when a smart device is within range that are suitable for use in the invention comprise Near Field Communication (NFC) and Radio-Frequency Identification (RFID). A packaged good with an NFC label may be activated by tapping an NFC-enabled device in the proximity of the NFC label. A packaged good with a passive RFID label may be activated when an RFID reader is brought into proximity of the RFID label. The encoded label may comprise Bluetooth Low-Energy™ or BLE™. In this embodiment, a BLET encoded label may be placed on a packaged good, with or without a shielded sticker. In the case where the shielded sticker is added on top of the label, the user can remove the shielded sticker, exposing the BLE™ label, which label can then be activated by electromagnetic induction such as NFC and RFID, or it may harvest energy from surrounding electromagnetic waves to produce a signal and communicate with a reader. In one embodiment, where the shielded sticker is not included, the BLE™ label can record physical properties of the packaged goods or its environment, including rotation, weight, movement, temperature, moisture, and light.

Other passive encoded labels that can be used in the invention comprise Quick Response (QR) codes which are matrix barcodes that are machine-readable optical labels and that contain information about the item to which the QR code is attached. Such encoded labels are not capable of activating and transmitting information and are “read-only.”

An active encoded label can transmit information to a smart device without the need for external power because a power source such as a battery is incorporated into the encoded label. In one embodiment, Bluetooth™ active devices may be used as the encoded label. The Bluetooth™ enabled label may be placed on the packaged good and shielded by a sticker. The user can remove the sticker, exposing the Bluetooth™ label, which the label can then be read by a Bluetooth™ reader.

Acoustics may be used as either a physical label or voice recognition signature by capturing and recording an acoustic signal that can be paired with a smart listening device such as a smartphone, Echo™ or Alexa™. The acoustic signal may be created by for example the tearing of an encoded label off a packaged good, cap opening, or a verbal command.

In most cases, activation of the encoded label occurs when a smart device reads encoded information from the encoded label, which encoded information is transmitted by the smart device to a cloud-based platform. Encoded label information contains information that can be used to identify the contents of the packaged good and also to identify the user of the system to whom the packaged good was sent. The smart device can also transmit non-encoded information that it determines at the time of activation of the encoded label. Such non-encoded information may comprise date, time, geolocation of package opening/use, as well as other physical properties of the packaged good or its surroundings (weight, rotation, opening, temperature, light, moisture, etc.).

In one embodiment, placement of the encoded label on the packaged goods will not obstruct any important governing information and instructions. In one embodiment, the encoded labels are covered with a shield (e.g., metallic aluminum shield) to block any signals that would cause unintentional activation/recording of events. The shield can be removed either by peeling or tearing to expose the encoded label and allow activation. In one embodiment, the encoded labels are coded for different or additional purposes beyond recording an event, such as directly linking the user to different features or components of the platform (including but not limited to data view, educational material, surveys, incentives, and communication with their provider).

The invention further comprises a platform comprising a database and programmable processor, a label encoding interface, a user portal, an admin/provider portal, a communication link and a memory. In one embodiment, the platform comprises a downloadable software program resident on a personal computer, or that may be accessed as software-as-a-service and resident on a server. The platform may also be accessible through an app resident on a smart device. The platform may further provide educational information in the nature of blogs, videos and articles. The platform may allow users to post information. In one embodiment, the platform configures data and information to be synced with the user's authorized apps and/or devices. The platform can also be customized to be embedded within other platforms and/or portals. In one embodiment, the platform is resident on a single server. In one embodiment, the platform is resident on a plurality of servers.

The label encoding interface allows authorized providers/users to prepare encoded labels to be attached to a packaged good, create or modify a provider profile, create or modify a user profile, add or modify specific goods associated with a user profile, define or modify criteria/thresholds for smart alerts, or send a key and/or links to users and providers for activating their profile. In one embodiment, encoded labels are prepared by the label encoding interface by encoding a label with a unique identifier, such as a serial number, which associates specific encoded labels with specific packaged goods and/or specific users.

Encoded labels can be attached to the packaged good by a variety of providers such as the manufacturer of the goods in the package or service providers who send the packaged good to a user. In one embodiment, the encoded label is attached to the packaged good by the user.

Encoded labels can be prepared by various parties. Encoded labels can be prepared by the same entity which attaches the encoded label to the packaged good, or by a different entity which provides the encoded label to an entity which attaches the encoded label to the packaged good. The encoded labels can be assigned to a packaged good substantially at the time they are attached to the packaged good or at a different time point. In one embodiment, encoded labels are prepared by a manufacturer before delivering the goods or packaged goods to a separate entity for shipment to a user. In one embodiment, encoded labels are prepared by the package manufacturer before delivering the goods or packaged goods to a separate entity for packaging and/or shipment to a user, such as a service provider, who then attaches the encoded labels to the packaged good. In one embodiment, encoded labels are prepared by a user who provides the encoded label to a manufacturer. In one embodiment, encoded labels are prepared by a user who provides the encoded labels to a service provider. In one embodiment, encoded labels are prepared by a user upon receipt of a packaged good. In one embodiment, encoded labels are attached to the packaged goods by the user upon receipt of the packaged goods. In one embodiment, encoded labels are prepared by a manufacturer who manufactures a good or a service provider who sends a packaged good to a user, and the user attaches the encoded label to the packaged good.

In one embodiment, the encoded labels comprise a unique identifier, such as a serial number, which allows for specific encoded labels to be associated with specific packaged goods and/or specific users in the database. The encoded labels may be provided by a manufacturer and attached to a specific packaged good. In this embodiment, when a specific encoded label is activated (also called a “tap”) by a user, the encoded information in the encoded label is transmitted by the user's smart device to the cloud-based platform. The cloud-based platform searches the database to determine if the encoded label exists and is associated with a user of the platform. If yes, the activation of the encoded label is recorded. If the encoded label does not exist in the database, the user is presented with a list of available medications or goods. The user associates the specific encoded label with a medication or good and the system acknowledges that the medication or good has been associated with the specific encoded label. The user may at that time also record a “tap,” indicating the recording of the event.

The invention further comprises a plurality of users, each user having an account stored in the memory of the platform and storing user information. The users are the consumers of the goods in the packages. The user portal allows users who are external of the platform to sign up for an account on the platform and store information on the platform for use in connection with the system and method of the invention. Users can activate a profile with a unique key provided to them by the platform administrator. User information may include username, password, personal information, preferences, types of goods purchased, recommended frequency of use of the purchased goods if any, providers of goods, etc. The platform may be programmed to sync with the user's apps and devices. Users for example may store personalized reminders of appointments and schedules of events and goods refill dates. The user may also store additional data such as health measures, attitudes, lifestyle and other experiences. These additional data may be linked to specific events. The user may generate a calendar that displays these data.

In one embodiment, the system and method may provide incentives to users who achieve goals as set forth in the user account. Such incentives may include but are not limited to cash rewards, coupons, rebates, gift cards, discounts, merchandise, memberships, motivational messages or downloadable files and games.

In one embodiment, the user portal and its features will be available in different languages for the user to set his or her preference. In one embodiment, users can add specific authorized contacts to their profile so that the contacts can access, view, and monitor data and information collected. In one embodiment, users can verify authenticity of the packaged good upon activation of the label.

The invention further comprises a plurality of providers who are external of the platform, each provider having an account stored in the memory of the platform and storing provider information. Providers provide packaged goods to users directly or indirectly. In one embodiment, a provider is a manufacturer of a good, who provides the packaged goods to a user via a distributor, wholesaler or a retailer. In one embodiment, a provider is the distributor, wholesaler or retailer who provides the packaged good to a user. In the healthcare setting, providers can be any professional recommending, prescribing, or authorizing the use of medications or the platform—including clinical or managed care physicians, nurse practitioners, physician assistants, pharmacists, nurses, and case managers. The admin/provider portal allows providers of packaged goods to sign up for an account on the platform and store information on the platform for use in connection with the system and method of the invention. Providers can activate a profile with a unique key provided to them by the platform administrator. Providers can access, view and monitor user information from users that use their packaged goods. Such information can be viewed at a user-level or an aggregate-level of all users and can be filtered by various parameters, including but not limited to date, time and event. Providers can for example search for specific events and can communicate with users through the platform by sending notifications and alerts to users who deviate from expected or predefined thresholds or outcomes based on usage of the provider's packaged goods. Providers may also use other methods to communicate with users, such as cell phones or email addresses, to communicate through SMS or email. In one embodiment, communication with the user requires user consent.

The platform further comprises a plurality of algorithms that are run by the database and programmable processor and that may activate certain actions based on information received from the smart device of a user.

The invention further comprises predetermined instructions associated with the various users. In one embodiment, the predetermined instructions are determined by one or more of the various providers. The predetermined instructions may be executed based upon information received (or not received) from an activated encoded label. For example, a provider may provide instructions in association with a user account relating to a specific encoded label based upon receipt of information that the encoded label was activated, presumably by the user. If the time of activation falls within a predetermined parameter, such as a certain time range, no instruction may be provided, or an instruction may be provided, including but not limited to take no action, to provide positive reinforcement or to link users to relevant resources. However, if the activation exceeds a predetermined threshold from the predetermined parameter, such as the time of activation exceeds a predetermined threshold for deviation from the predetermined parameter by falling outside of the certain time range, a message may be automatically sent by the platform through the communications link to the user, for example reminding the user of the next time to open the packaged good and activate the encoded label to consume the packaged goods. In one embodiment, where the packaged good is a medication, the message may instruct the user that it is time to consume the packaged goods. (e.g., take medication). Similarly, failure to receive information within a certain time range may be interpreted as meaning that the encoded label was not activated because the packaged good was not opened and a message may be automatically sent by the platform through the communications link to the user, for example reminding the user to open the packaged good and activate the encoded label to consume the goods. In one embodiment, where the packaged good is a medication, the message may instruct the user that it is time to consume the packaged goods. Reminders to users may be sent by various methods, including but not limited to in-App alerts, push notifications, SMS text messages, email, mail, phone call, chatbot, fax, letters, and visits. The algorithms can escalate the degree of outreach to the user depending on the variation of the user's behavior from predefined thresholds and frequency of how often these thresholds are crossed.

In one embodiment, a treatment plan or schedule can be entered in the user account either by the provider or entered by the user him/herself at the time that the medication or good is added into the user profile account. In one embodiment, the treatment plan or schedule may be changed in the user account, allowing for the user/patient to continue using the same label even if the treatment plan or schedule has been changed.

In one embodiment, machine learning analytics and predictive modeling are applied to identify user characteristics and behaviors predictive of certain data and outcomes, giving insights to providers on how to personalize user engagement for proactive outreach and communication. For example, in the context of medication adherence, the algorithms can proactively identify users who are more likely to be non-adherent and thus more likely to experience associated complications due to nonadherence, and help providers intervene prior to the onset of poor outcomes due to non-adherence.

In one embodiment, the app will have native compatibility with the encoded labels to automatically record an event when an encoded label is read by the smart device. If the encoded label is not read within the predefined range of time, the platform will recognize the lack of signal and launch a notification (such as SMS message, email, push notification, reminders, etc.) that will direct the user to the user interface to report event status or record the event. In one embodiment, the smart device transmits information read from an encoded label to the platform only when the app or software is open. In one embodiment, for example where the encoded label is an active encoded label, the smart device opens an app or software when information is read from the encoded label and transmits the information to the platform.

In one embodiment, the platform transmits user's data and information to a provider's existing electronic databases and/or records. In the context of healthcare, this can include electronic health records (EHRs).

The invention yet further comprises a plurality of user devices, each user device comprising an app or web-based software that is enabled to receive information from the encoded label on the packaged good. The type of enablement will depend on the type of encoded label. Examples of suitable smart devices include but are not limited to smartphones, tablets, notebooks and laptop computers.

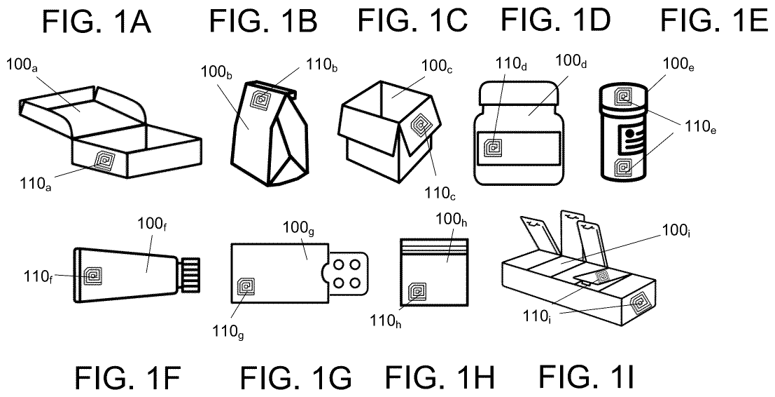

Turning to the figures, FIGS. 1A-1I depict a variety of packaging 100i and exemplary placement of encoded labels 110i as used in the invention.

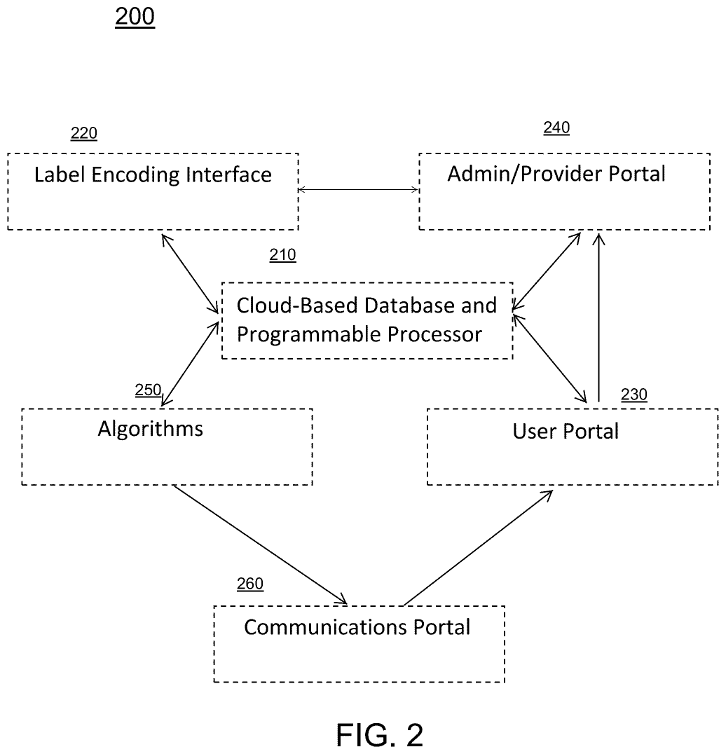

FIG. 2 depicts a schematic of a platform 200 according to one embodiment of the invention. Platform 200 comprises a database and programmable processor 210; a label encoding interface 220; a user portal 230; an admin/provider portal 240; a plurality of algorithms 250; and a communications portal 260.

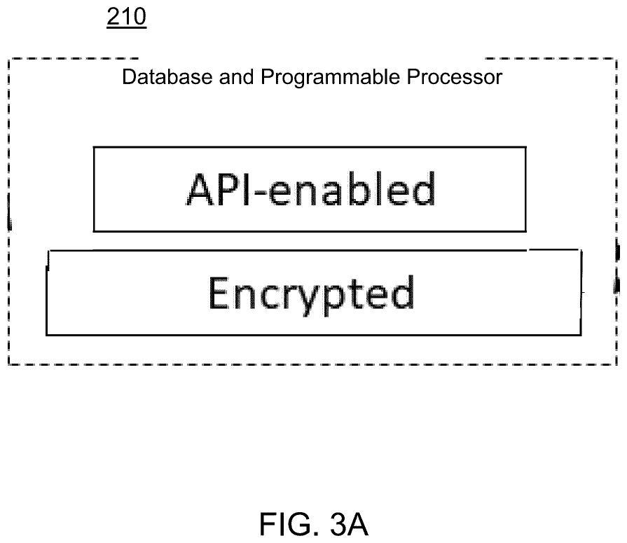

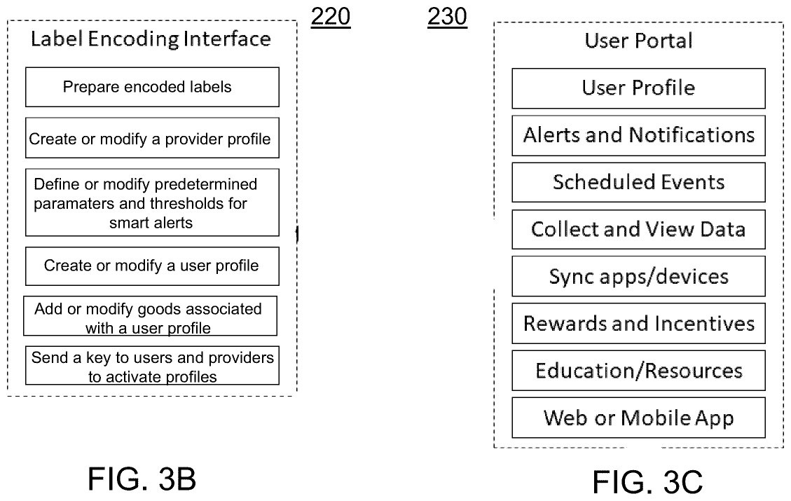

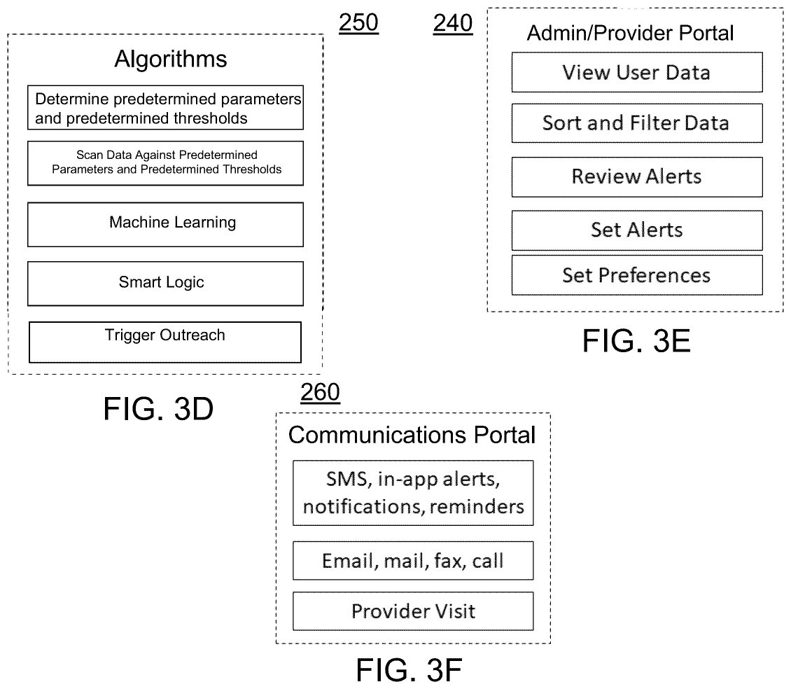

FIGS. 3A-3F depict schematics of database and programmable processor 210; label encoding interface 220; user portal 230; admin/provider portal 240; plurality of algorithms 250; and communications portal 260 according to one embodiment of the invention. In FIG. 3A, database and programmable processor 210 is API-enabled and encrypted according to one embodiment of the invention. In FIG. 3B, label encoding interface 220 can be accessed by users and providers; links users and providers; encodes smart labels to each unique user; modifies a user's schedule; set and/or modify triggers for execution of algorithms; set and/or modify thresholds for determination of non-adherence; and provide an administration center where settings for platform 200 can be set. In FIG. 3C, user portal 230 allows users to set a user profile; set alerts and notifications; set scheduled events; collect and view data; sync apps and devices with platform 200; view rewards and incentives provided by platform 200; access education and resources; and allows access through a web-based or mobile app. In FIG. 3D, a plurality of algorithms 250 comprise routines and/or programs that are executable by database and programmable processor 210 that calculate thresholds for users; scan user data; trigger communications outreach to users; and determine various parameters based on user data by use of smart logic, AI, or machine learning. In FIG. 3E, admin/provider portal 240 allows providers to view user data; sort and filter user data; review alerts triggered by user data; set alerts for users; and set preferences. In FIG. 3F, communications portal 260 allows providers and users to send and receive communications including but not limited to SMS, in-app alerts, notifications and reminders; email, postal or private mail, faxes and telephone calls using the Plain Old Telephone Service (POTS); and set up provider visits.

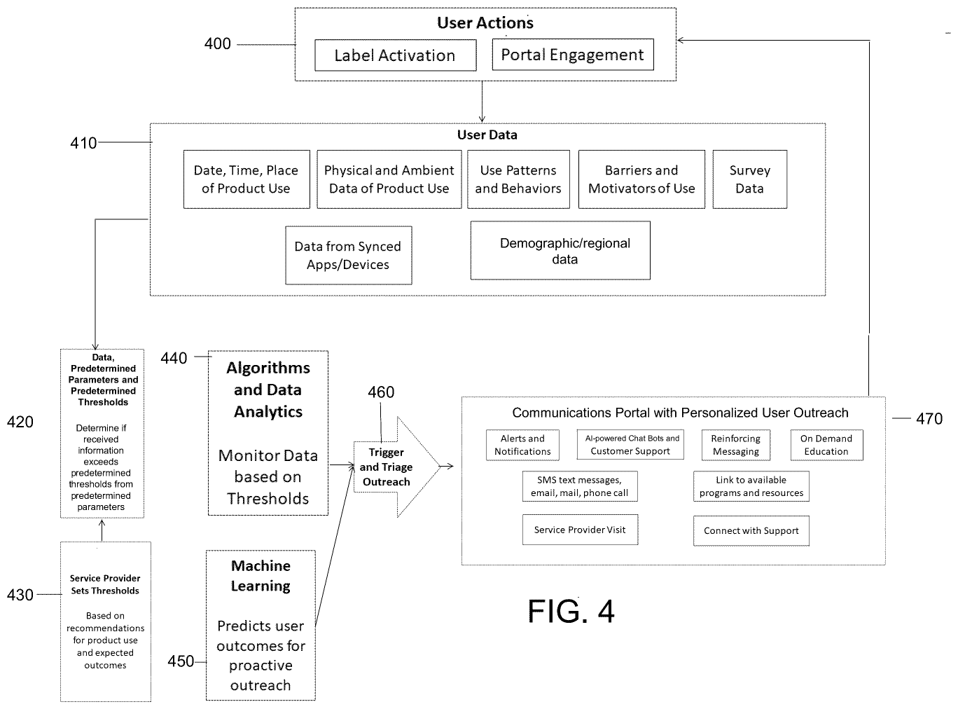

FIG. 4 depicts a flow scheme of the execution of exemplary algorithms 250 resident in database and programmable processor 210 of platform 200 that can be executed by database and programmable processor 210. At 400, a plurality of algorithms 250 can be executed based upon user actions that are detected through label activation, portal engagement or any combination of label activation and portal engagement. At 410, providers access user data including but not limited to date, time and place of packaged goods use; physical and ambient data of packaged goods use; use patterns and behaviors of user; barriers and motivators of use of the user; survey data of the user; data synced from user's apps and devices; and other demographic and regional data that may or may not be personally identifiable. At 420, algorithms 250 identify deviance from expected outcomes and user consumption behavior based on user data and determine if the deviance from the predetermined parameter exceeds the predetermined threshold. At 430, providers input expected thresholds based on recommendations for goods use and expected outcomes for various users. At 440, user outcomes are predicted using AI/machine learning. At 450, data relating to thresholds for individual users are monitored. At 460, communications and outreach to individual users are instigated if certain thresholds are triggered. At 470, the types of outreach may take the form of one or more communications, including but not limited to alerts and notifications sent through communications portal 260; AI-powered chatbots (conversation interfaces) and customer support; reinforcement of messaging; on demand education; SMS text messages, email, postal or private mail and phone calls using the POTS: providing links to available programs and resources; physical or virtual visits by providers; and connection with other support.

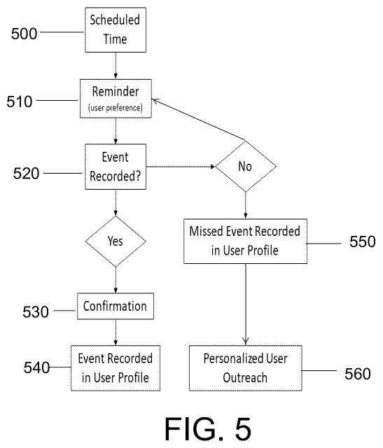

FIG. 5 depicts a flow scheme of exemplary actions that may be taken upon confirmation of an event recorded on a user profile according to one embodiment of the invention. A scheduled time is set for a certain event at 600. At 510, a reminder of the event is sent to the user. At 520, the platform 200 determines if the event occurred in accordance with the scheduled time. If yes, at 530, occurrence of the event is acknowledged and at 540 occurrence of the event is recorded in the user profile. If no, the user is sent another reminder at 510. The missed event is recorded in the user profile at 550 and at 560 a provider undertakes personal outreach to the user.

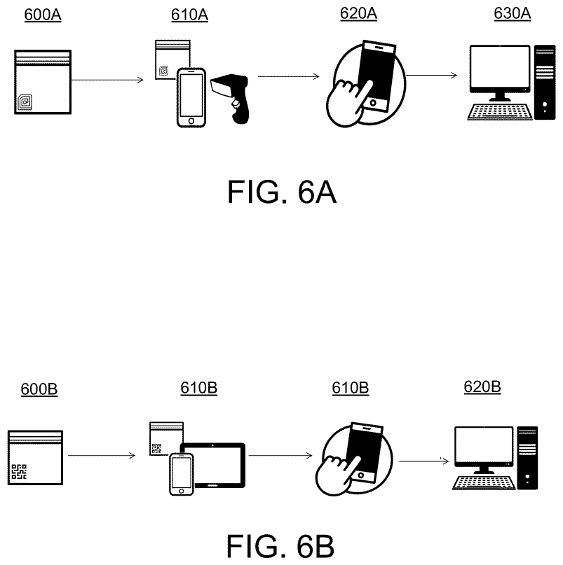

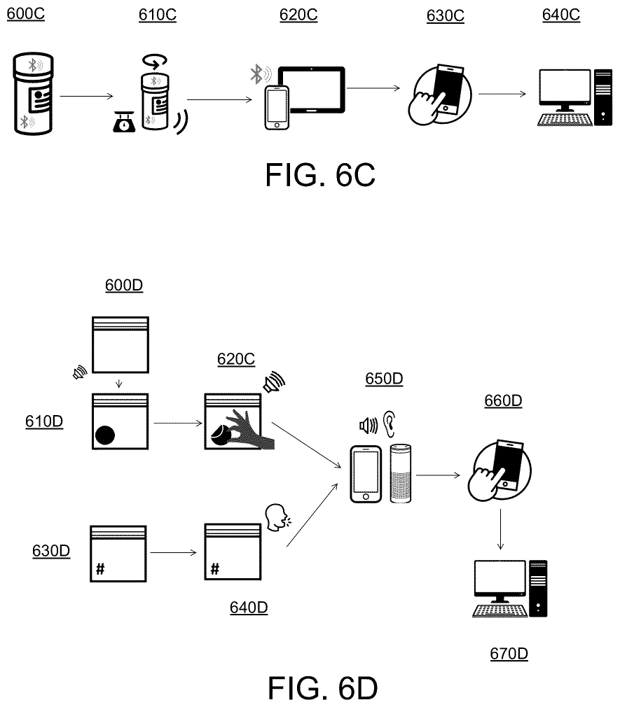

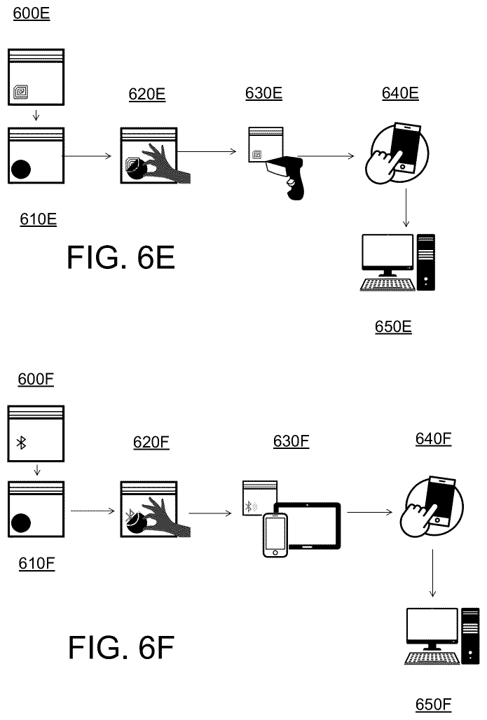

FIGS. 6A-6F depict activation schematics of various encoded labels as used according to one embodiment of the invention. FIG. 6A depicts activation of an encoded label with NFC. At 600A, an encoded NFC label is attached to or embedded in a package good. At 610A, the encoded NFC label is read by an NFC reader or an NFC-enabled device by the user. At 620A, information read from the encoded NFC label indicating event confirmation is transmitted to platform 200. At 630A, event confirmation is stored in the user profile. FIG. 6B depicts activation of an encoded label with a QR code. At 600B, an encoded QR code label is attached to or embedded in a packaged good. At 610B, the encoded QR code label is scanned by a QR code reader or a QR code-enabled device of the user. At 620B, information indicating event confirmation is transmitted to platform 200. At 630B, event confirmation is stored in the user profile. FIG. 6C depicts activation of an encoded label with Bluetooth™. At 600C, an encoded Bluetooth™ label is attached to or embedded in a packaged good. At 610C, package use and/or movement/rotation/weight of the packaged good is recorded by a Bluetooth™ reader or Bluetooth™-enabled smart device. At 620C, the encoded Bluetooth™ label transmits the physical parameter data to a Bluetooth™ reader or a Bluetooth™-enabled device by the user. At 630C, information read from the encoded Bluetooth™ label indicating event confirmation is transmitted to platform 200. At 640C, event confirmation is stored in the user profile. FIG. 6D depicts two methods of activation of an encoded label with acoustic label. In the first method, at 600D, an encoded acoustic label is attached to or embedded in a packaged good. At 610D, a shield is placed over the encoded acoustic label. At 620D, the user removes the shield which sound triggers an acoustic signal. Other suitable acoustic signals include but are not limited to the sound created by the opening of a bottle cap or the sound created by the tearing open of a package. In the second method, at 630D an acoustic key code label package is attached to or embedded in a packaged good. At 640D, the user speaks the key code. For both methods, at 650D the acoustic signal is captured by a smart listening device. At 660D, information detected by the smart listening device indicating event confirmation is transmitted to platform 200. At 670D, event confirmation is stored in the user profile. FIG. 6E depicts activation of an encoded label with an RFID chip. At 600E, an encoded RFID chip label is attached to or embedded in a packaged good. At 610E, a shield is placed over the encoded RFID chip label. At 620E, the shield is removed to expose the encoded RFID chip label. At 630E, the encoded RFID chip label is scanned by an RFID reader or a RFID reader-enabled device by the user. At 640E, information read from the encoded RFID chip label indicating event confirmation is transmitted to platform 200 or alternatively is stored in an RFID application on the user's device. At 650E, event confirmation is stored in the user profile. 6F depicts activation of an encoded label with shielded Bluetooth™ or BLE™. At 600F, an encoded Bluetooth™ or BLE™ label is attached to or embedded in a packaged good. At 610F, a shield is placed over the encoded Bluetooth™ or BLE™ label. At 620F, the shield is removed to expose the encoded Bluetooth™ or BLE™ label. At 630F, the encoded Bluetooth™ or BLE™ label is scanned by a Bluetooth™ reader or a Bluetooth™ reader-enabled device by the user. At 640F, information read from the encoded Bluetooth™ or BLE™ label indicating event confirmation is transmitted to platform 200. At 650F, event confirmation is stored in the user profile.

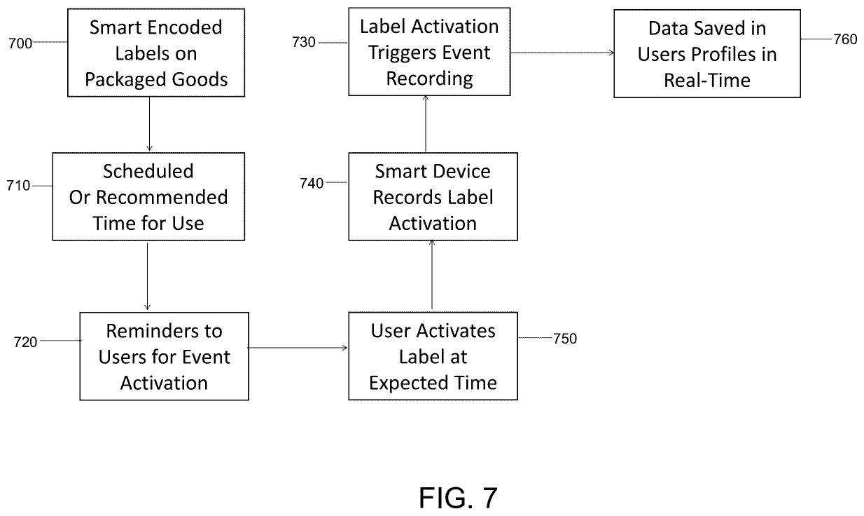

FIG. 7 depicts a flow scheme of the method according to one embodiment of the invention. At 700, an encoded label is embedded in or placed on a packaged good by a provider who obtained the encoded label from platform 200. At 710, a scheduled or recommended time for use of the packaged good is determined. At 720, a reminder is sent to a user of the packaged good to activate the label, thus indicating opening of the packaged goods. At 730, the user opens the packaged goods, indicating usage of the contents of the packaged goods. At 740, event activation is detected by the encoded label. At 750, event activation is recorded. At 760, data regarding event activation are recorded in a user profile in the platform.

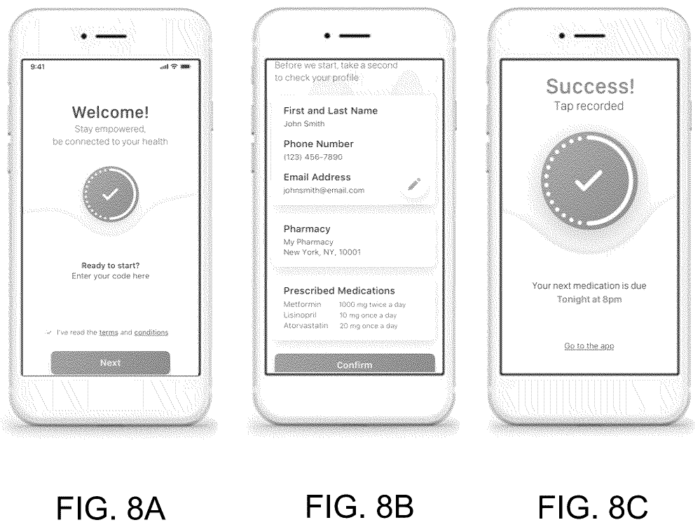

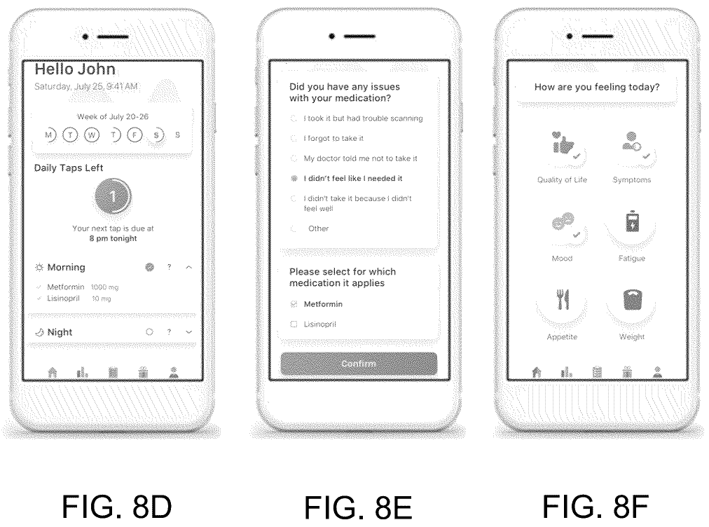

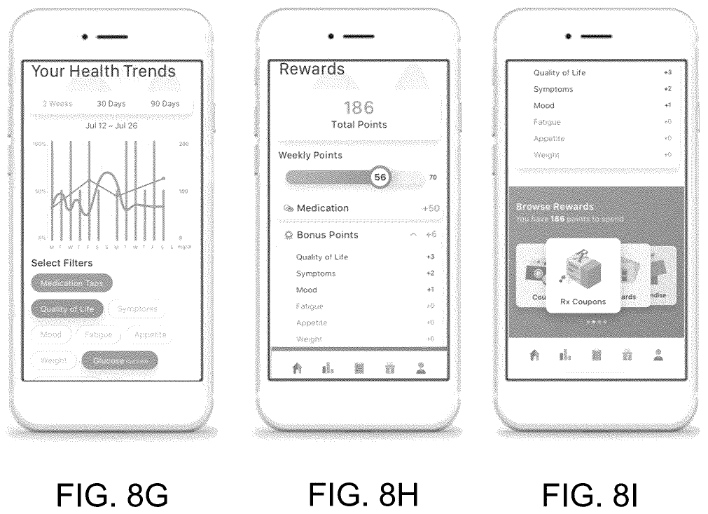

FIGS. 8A-8I depict mock-up screens depicting platform 200 as viewed through an app resident on a smartphone of a user of the platform according to one embodiment of the invention. FIG. 8A depicts a Welcome screen or Home page of the platform. FIG. 8B depicts user information as stored in the user profile. FIG. 8C depicts confirmation of a successful event activation. FIG. 8D depicts reminders of upcoming events. FIG. 8E depicts requests for user input regarding events. FIG. 8F depicts a questionnaire to gather data regarding user use of the goods in the package. FIG. 8G depicts gathered data in graph form. FIGS. 8H and 8I depict rewards awarded to the user.

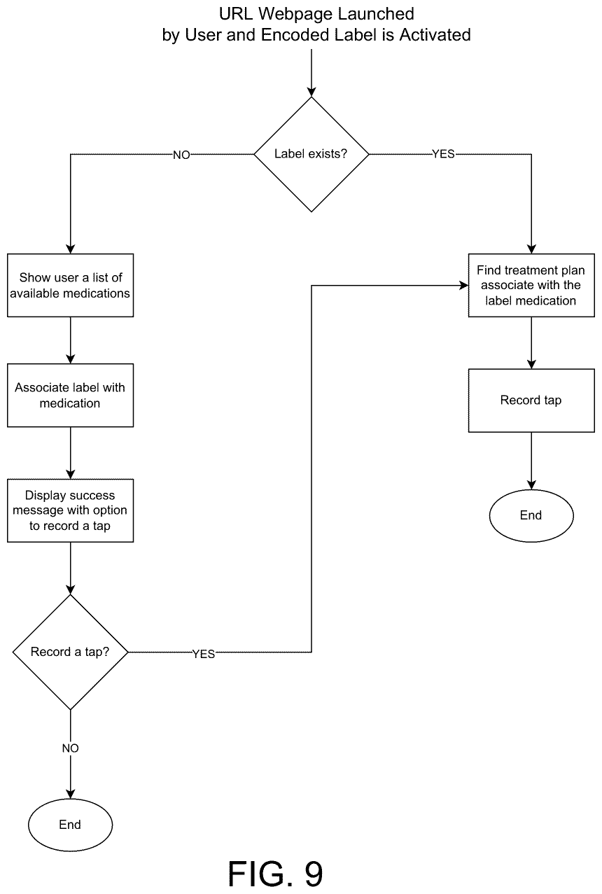

FIG. 9 depicts one embodiment of the invention. In this embodiment, at 910 a specific encoded label is activated or triggered (also called a “tap”) by a user. At 915, the cloud-based platform determines if the specific encoded label is stored in the database. If the specific encoded label is not stored in the database, at 920 the user is presented by the cloud-based platform with a list of medications or consumable goods. At 925, the user selects a medication or consumable good to associate with the specific encoded label. At 930, the cloud-based platform indicates success to the user and offers the user the option to record encoded label activation. At 935, if the user opts not to record activation, no further action is taken. At 935, if the user opts to record activation, at 940 the cloud-based platform finds the treatment plan or schedule associated with the medication or good that is associated with the specific encoded label. At 945, the cloud-based platform records that the encoded label has been activated. At 950, the routine ends.

The foregoing description of various embodiments of the invention has been presented for purposes of illustration and description. It is not intended to be exhaustive or to limit the invention to the precise form disclosed and, obviously, many modifications and variations are possible. While the invention has been described in terms of medication adherence, the invention is not limited to such a purpose. Modifications and variations that may be apparent to a person skilled in the art are intended to be included within the scope of the invention as defined by the accompanying claims.

What is claimed is:

1. A method for real-time tracking, monitoring, and engagement of users with regard to opening of packaged goods and personalization of user engagement through the use of encoded labels and a cloud-based platform, comprising:
providing an encoded label, wherein the encoded label comprises encoded information;
thereafter receiving the encoded information from the encoded label, wherein the encoded information is transmitted by a smart device;
comparing the received encoded information to predetermined parameters that have been stored in a database, wherein the predetermined parameters comprise sufficient information to identify contents of a packaged good to which the encoded label is attached, a user of the contents of the packaged good or a combination of contents of the packaged good and the user;
determining if the received encoded information deviates from one or more of the predetermined parameters that have been stored in the database;
determining if any action should be taken depending on the deviation of the received encoded information from one or more of the predetermined parameters that have been stored in the database;
thereafter taking one or more actions depending on the deviation of the received encoded information if it is determined that action should be taken; and
recording the received encoded information and the one or more actions taken, if any, in the database.

2. The method of claim 1, wherein the recorded received encoded information and the one or more actions taken are made accessible to a provider.

3. The method of claim 2, wherein the provider comprises a provider of medication.

4. The method of claim 3, wherein the medication comprises a prescription medication.

5. The method of claim 3, wherein the predetermined parameters comprise a recommended dosing regimen of the prescription medication.

6. The method of claim 1, wherein the action that is taken if the received encoded information deviates from the predetermined parameters comprises one or more communications sent to the user.

7. The method of claim 6, wherein the one or more communications comprise push notifications, alerts, SMS text messages, email, mail, telephone call, chatbot, fax, letter or in-person visit.

8. The method of claim 7, wherein one or more of the communications are sent automatically through a communications portal.

9. The method of claim 7, wherein the encoded label comprises an encoded NFC label, an encoded RFID label, an encoded QR code label, an encoded Bluetooth™ label, an encoded BLE label, or an encoded acoustic label.

10. The method of claim 9, wherein the acoustic encoded label comprises sound generation when the package bearing the acoustic encoded label is opened.

11. The method of claim 10, wherein the acoustic encoded label comprises voice signals given when the package bearing the acoustic encoded label is opened.

12. The method of claim 1, wherein the cloud-based platform determines if the specific encoded information identifying the specific encoded label is stored in the database, wherein if the specific encoded label is not stored in the database, the user:
is presented by the cloud-based platform with a list of medications; and
the user selects a medication to associate with the specific encoded label.

13. A programmable computer-based system for real-time tracking, monitoring, and engagement of users with regard to opening of packages and personalization of user engagement through the use of encoded labels and a cloud-based platform, comprising:
a database and programmable processor;
a label interface;
a user portal;
an admin/provider portal;
a plurality of algorithms; and
a communications portal,
wherein the database and programmable processor is API-enabled to receive information, which received information has been read from one or more encoded labels placed on a packaged good,
wherein the user portal provides access to the system by a plurality of users and stores user information comprising a user profile,
wherein the admin/provider portal provides access to the system by a plurality of providers and permits one or more providers to access user information stored in the user portal,
wherein the label interface allows for the association of a specific medication with a specific encoded label, wherein the encoded labels are encoded with information sufficient to identify the specific encoded label, wherein the label interface further stores predetermined parameters related to the contents of a packaged good to which the specific encoded label is attached,
wherein one or more of the plurality of algorithms are executable by the database and programmable processor based upon the received information from the one or more user devices, wherein the one or more plurality of algorithms determines whether received information deviates from the predetermined parameters,
wherein the communications portal transmits one or more communications to one or more users based on one or more results of the execution of the one or more of the plurality of algorithms if the deviation of the received information from predetermined parameters exceeds predetermined thresholds,
wherein the one or more communications comprise push notifications, alerts, SMS text messages, email, mail, telephone call, chatbot, fax, letter or in-person visit.

14. The system of claim 13, wherein the communications portal automatically sends out one or more communications.

15. The system of claim 13, wherein the predetermined parameters comprise the recommended dosing regimen of a prescription medication.

16. The system of claim 15, wherein the information read from the one or more encoded labels indicates whether a packaged good containing the prescription medication has been opened.

17. The system of claim 16, wherein the encoded label comprises an NFC encoded label, an encoded QR code label, a Bluetooth™ encoded label, an acoustic encoded label, an RFID encoded label, or a Bluetooth Low Energy™ encoded label.
